(19) United States
(12) Patent Application Publication
(10) Pub. No.: US 2025/0268973 A1
(43) Pub. Date: Aug. 28, 2025

(54) GINGER EXTRACT POLYSACCHARIDE COMPOSITION AND METHOD OF SUPPORTING THE IMMUNE SYSTEM

(71) Applicant: Kodimule Shyam PRASAD, Bangalore (IN)

(72) Inventor: Kodimule Shyam PRASAD, Bangalore (IN)

(21) Appl. No.: 19/060,659

(22) Filed: Feb. 22, 2025

Related U.S. Application Data
(60) Provisional application No. 63/557,527, filed on Feb. 24, 2024.

(51) Int. Cl. A61K 36/9068; A61P 37/04

(52) U.S. Cl. CPC A61K 36/9068; A61P 37/04

(57) ABSTRACT

The invention provides an immunomodulatory composition comprising an extract of Zingiber officinale root containing one or more biologically active polysaccharides. The composition finds use in methods of supporting and maintaining health and the immune system.

### CROSS-REFERENCE TO RELATED APPLICATIONS

This application claims the benefit of provisional application No. 63/557,527, filed Feb. 24, 2024, the entire contents of which are incorporated herein by reference for all purposes.

### FIELD OF THE INVENTION

The invention generally relates to botanical extracts. More particularly, the invention relates to a composition comprising a ginger extract containing one or more polysaccharides and methods of using the composition in supporting and maintaining health and the immune system.

### BACKGROUND

A healthy immune system can respond to foreign invaders through immunomodulation. The immune system plays a critical role in preventing and ridding the body of disease. A properly functioning immune system can respond to foreign invaders, such as viruses, fungi and bacteria, by removing them from the body. However, in dysregulated immune systems, the immune system can become overactive, leading to numerous diseases. The strategy to activate or suppress the immune system by natural or synthetic immunomodulators has become increasingly popular in the treatment of various types of infections and cancers.

Plant polysaccharides play a key role in many aspects of plant biology, including providing a defense against pathogens and stress. Plant polysaccharides are increasingly being utilized in dietary supplements, animal health products, skincare applications, and to progress a range of innovative pharmaceutical research projects. Trends in current research suggest the application of polysaccharide-based therapeutics in human nutrition and health has immense potential.

### SUMMARY OF THE INVENTION

The invention provides a composition comprising a ginger extract that contains one or polysaccharides. The composition finds use in methods of maintaining, supporting and stimulating the immune system, and supporting health overall. The invention also provides methods of making the inventive composition.

In some aspects, the invention provides a method of stimulating or supporting the immune system, comprising administering to a subject in need thereof an effective amount of the inventive composition.

### BRIEF DESCRIPTION OF THE DRAWINGS

FIG. 1 shows the effect of an embodiment of the inventive composition on viability and morphology of RAW264.7 cells. The RAW 264.7 cells were treated with various concentrations of the composition for 24 h and cell viability was measured. Cell viability is expressed as a relative value compared to that of vehicle-treated cells, which is set to 100% (A). Cell morphology was observed under phase-contrast microscope (B).

FIG. 2. shows the effect of an embodiment of the inventive composition on NO production in RAW264.7 cells. Cells were treated with two different concentrations (50 and 100 μg/mL) of the composition or LPS (1 μg/mL) for 24 h prior to measure nitrite level. The data arc expressed as the mean±SEM. **P<0.01, ***P<0.001, ##P<0.001 as compared to control.

FIG. 3. shows the effect of an embodiment of the inventive composition on phagocytic activity of treated macrophages. The data are expressed as the mean±SEM. **P<0.01, ***p<0.001, ##P<0.001 as compared to control.

FIG. 4. shows the effect of an embodiment of the inventive composition on increases in the cytokine expression levels of proinflammatory cytokines in RAW 264.7 cells. RAW 264.7 cells were treated with the composition at a concentration of 50 and 100 μg/ml or LPS (1 μg/mL) for 24 h and cytokines levels were measured using ELISA for TNF-α (A), IL-6 (B). The data are expressed as the mean±SEM. **P<0.01, *** P<0.001, ##P<0.001 as compared to control.

FIG. 5. shows the effect of an embodiment of the inventive composition on LPS-induced iNOS (A) and COX-2 protein expression levels (B). Raw 264.7 cells were pre-treated with the composition for 1 h before incubation with LPS (1 μg/ml) for 24 h. Cell lysates were prepared and analyzed for iNOS and COX-2 protein expression levels by Western blotting.

FIG. 6. shows the effect of an embodiment of the inventive composition on the protein expression of TLR4 signaling pathways in RAW264.7 cells.

FIG. 7. shows the effect of an embodiment of the inventive composition on protein expression related to the MAPK signaling pathway in RAW264.7 macrophages. The levels of protein expression related to the MAPK signaling pathway were determined by Western blot and quantitative analysis of phosphorylated JNK and ERK. Treatment groups showed significant differences, P<0.05.

### DETAILED DESCRIPTION

The invention provides a composition comprising a ginger extract containing one or more polysaccharides. The inventive composition finds use in methods of supporting and maintaining health, including methods of supporting and maintaining the immune system.

In some aspects, the invention provides a method of using the composition for one or more of supporting and maintaining the immune system, promoting immune system health, and stimulating the immune system. These methods can be practiced by administering an effective amount of the inventive composition to a subject in need thereof. The inventive composition can also be practiced in methods of promoting health by administering an effective amount of the inventive composition to a subject. The term “subject” as used herein refers to mammals such as humans and non-human primates, as well as livestock and companion and laboratory research animals.

The subject can be a human subject desirous of supporting and maintaining the immune system, promoting immune system health, stimulating the immune system, or combinations thereof. The subject can be at risk of being exposed to one or more pathogens, can have been exposed to one or more pathogens, or be infected with one or more pathogens. In some embodiments, the subject is not infected with a pathogen and the composition is administered to the subject to prevent, or decrease the likelihood of, the subject of becoming infected with one or more pathogens. As used herein, the terms “decrease,” “decreases,” “decreasing,” and the like, refer to any measurable decrease in a parameter as a result of the administration of the inventive composition compared to control conditions. The composition can be administered to a subject that is not infected with a pathogen, wherein administering the composition to the subject decreases the length of time the subject is infected with a pathogen after infection with the pathogen occurs. The composition can be administered to a subject that is not infected with a pathogen, wherein administering the composition to the subject decreases the severity of symptoms associated with a pathogenic infection after infection with a pathogen occurs. In practicing the methods disclosed herein, the symptoms of a pathogenic infection can include, but are not necessarily limited to, coughing, fever, chills, headache, nasal congestion, runny nose, sneezing, muscle aches, fatigue, irritated respiratory tract, irritated throat, diarrhea, or combinations thereof. The subject can be infected with a pathogen, wherein administering the composition to the subject decreases the length of time the subject is infected with the pathogen. The subject can be infected with a pathogen, wherein administering the composition to the subject decreases the severity of the symptoms associated with the pathogenic infection. The subject can be infected with a pathogen, wherein administering the composition to the subject decreases the length of time the subject experiences the symptoms associated with the pathogenic infection.

In practicing the methods disclosed herein, the pathogen can be a virus, bacteria, fungus, parasite, prion, or combinations thereof. In some embodiments, the pathogen is one or more pathogens associated with colds, flu, COVID, pneumonia, and respiratory syncytial virus (RSV) infection. The pathogen can be a virus, such as, for example, one or more of a rhinovirus, an influenza virus (e.g., influenza A virus), a coronavirus (e.g., SARS-COV-2 virus) and RSV. In some embodiments, the pathogen is a pathogen associated with pneumonia, including, but not necessarily limited to, streptococcus pneumoniae, haemophilus influenzae, staphylococcus aureus, legionella pneumophila, mycoplasma pneumoniae, chlamydia pneumoniae, or combinations thereof.

The composition can be administered systemically and/or locally. The composition can be administered locally, such as at the site of an infection or to a tissue site at risk of developing an infection, such as a wound, for example. Suitable administration routes for the composition include, but are not limited to, auricular, buccal, conjunctival, cutaneous, dental, endocervical, endosinusal, endotracheal, enteral, epidural, extra-amniotic, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal dental, intracoronary, intracorporus cavernosum, intradermal, intradiscal, intraductal, intraduodenal, intradural, intraepidermal, intraesophageal, intragastric, intravaginal, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravitreal, laryngeal, nasal, nasogastric, ophthalmic, oral, oropharyngeal, parentera, percutaneous, periarticular, peridural, perineural, periodontal, rectal, inhalation, retrobulbar, soft tissue, subarachnoid, subconjunctival, subcutaneous, sublingual, submucosal, topical, transdermal, transmucosal, transplacental, transtracheal, transtympanic, ureteral, urethral, vaginal, or combinations thereof. The composition can be administered by irrigation, drip, infusion, or topically by a dressing, patch, or bandage that is in contact with the composition. In a preferred embodiment, the composition is administered orally.

In some aspects, the dosage of the composition administered to a subject is determined by the body weight of the subject. The composition can be administered such that the dosage of the extract is between about 5 mg/kg b.w. and about 500 mg/kg b.w. The composition can be administered at a dosage that is about 5 mg/kg b.w., about 10 mg/kg b.w., about 20 mg/kg b.w., about 30 mg/kg b.w., about 40 mg/kg b.w., about 50 mg/kg b.w., about 60 mg/kg b.w., about 70 mg/kg b.w., about 80 mg/kg b.w., about 100 mg/kg b.w., about 120 mg/kg b.w., about 140 mg/kg b.w., about 150 mg/kg b.w., about 160 mg/kg b.w., about 180 mg/kg b.w., about 200 mg/kg b.w., about 220 mg/kg b.w., about 240 mg/kg b.w., about 260 mg/kg b.w., about 280 mg/kg b.w., about 300 mg/kg b.w., about 320 mg/kg b.w., about 340 mg/kg b.w., about 360 mg/kg b.w., about 380 mg/kg b.w., about 400 mg/kg b.w., about 420 mg/kg b.w., about 440 mg/kg b.w., about 460 mg/kg b.w., about 480 mg/kg b.w., or about 500 mg/kg b.w. As used in this disclosure, the term “about” means the stated amount, quantity or value, or that varies (plus or minus) by 20% of the stated amount, quantity or value.

The composition can be administered, one, two, three, four, five or more times. The administration schedule can be daily, weekly, monthly, or combinations thereof. For example, the composition can be administered two times per day for two months. In some embodiments, the composition is administered as needed.

In some aspects, the invention provides a method of making the inventive composition. The composition can be made by providing a ginger material and subjecting the ginger material to an extraction solvent to provide a ginger extract containing one or more polysaccharides. The ginger can be Zingiber officinale. The ginger material can be any part of the ginger plant, including the root, stem, leaves, flowers, seeds, or combinations thereof. In a preferred embodiment, the ginger material is ginger root.

Suitable solvents for obtaining the ginger extract of the invention include aqueous solvents, organic solvents, supercritical fluid solvents, or combinations thereof. Non-limiting examples of suitable solvents include water, aqueous alcohol, anhydrous alcohol, hexane, absolute acetone, aqueous acetone, methylethyl ketone, dichloromethane, diethyl ether, petroleum ether, chloroform, ethyl acetate, or combinations thereof. Suitable alcohols for use as the solvent include, but are not necessarily limited to, methanol, ethanol, isopropyl alcohol, or combinations thereof. In some non-limiting embodiments, the ginger extract is obtained by supercritical fluid extraction. The supercritical fluid extraction solvent can be, but is not necessarily limited to, carbon dioxide.

In some embodiments, the inventive composition comprises a ginger extract that is standardized for one or more polysaccharides. The extract can be standardized for one or more polysaccharides in an amount of at least 5% w/w, at least 10% w/w, at least 15% w/w %, at least 20% w/w, at least 25% w/w %, at least 30% w/w, at least 35% w/w, at least 40% w/w, at least 45% w/w or at least 50% w/w. The extract can be standardized for one or more polysaccharides in an amount of up to 5% w/w, up to 10% w/w, up to 15% w/w %, up to 20% w/w, up to 25% w/w %, up to 30% w/w, up to 35% w/w, up to 40% w/w, up to 45% w/w or up to 50% w/w.

The composition can comprise ginger extract containing one or more polysaccharides, wherein the extract is combined with one or more pharmaceutically acceptable excipients. The pharmaceutically acceptable excipient can be an artificial excipient. The excipient can be selected from the group consisting of binders, fillers, bulking agents, flow aids/glidents, disintegrants, lubricants, stabilizers, surfactants, and combinations thereof. Suitable excipients for use with the composition include, but are not limited to, those disclosed in the following publications, the entire contents of which are incorporated herein by reference for all purposes: Remington: The Science and Practice of Pharmacy, 19^{th} Ed (Easton, Pa.: Mack Publishing Company, 1995); Hoover, John E., Remington's Pharmaceutical Sciences, (Easton, Pa.: Mack Publishing Co 1975); Liberman, H. A. and Lachman, L., Eds., Pharmaceutical Dosage Forms (New York, N.Y.: Marcel Decker 1980); and Pharmaceutical Dosage Forms and Drug Delivery Systems, Seventh Ed (Lippincott Williams & Wilkins 1999).

The composition can be in any form suitable for administering the composition to a subject. The composition can be in the form of a powder, liquid, pill, tablet, pellet, capsule, thin film, solution, spray, syrup, linctus, lozenge, pastille, chewing gum, paste, vapor, suspension, emulsion, ointment, cream, lotion, liniment, drop, topical patch, buccal patch, bead, gummy, gel, sol, or injection. The composition can be formulated for oral administration. The composition can be provided as a drink. The drink can be a tea comprising the ginger extract of the invention. The tea can further include one or more teas obtained from tea leaves and/or herbs. In some embodiments, the composition is formulated as an effervescent powder or tablet. The composition can be formulated as a nutritional or dietary supplement containing minerals, vitamins, carbohydrates, proteins, botanical extracts, or combinations thereof.

In some embodiments, the composition is provided as a controlled, sustained, or extended release formulations known collectively as “modified release” formulations. Examples of non-limiting modified release formulations include, but are not limited to, those described in the following U.S. Patents, the entire disclosures of which are incorporated herein by reference for all purposes: U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,566. Modified release formulations and systems for use with the invention can include, for example, hydropropylmethyl cellulose, other polymer matrices, gels, permeable membranes, osmotic systems, multilayer coatings, microparticles, liposomes, microspheres, and combinations thereof.

The composition can further comprise one or more vitamins, one or more minerals, one or more extracts, one or more herbs, one or more amino acids, one or more proteins, one or more carbohydrates, one or more lipids, caffeine, one or more flavorings, one or more sweeteners, one or more preservatives, or combinations thereof. In some embodiments, the inventive composition is free of other agents having any biological or physiological effect when administered to a subject. For example, the composition can be free of any other agent that is effective in stimulating, supporting, maintaining, suppressing, or otherwise modulating the immune system. The inventive composition can consist of, or consist essentially of, the ginger extract disclosed herein. By “consisting of” is meant including, and limited to, whatever follows the phrase “consisting of.” Thus, the phrase “consisting of” indicates that the listed elements are required or mandatory, and that no other elements may be present. The phrase “consisting essentially of” means including any elements listed after the phrase and excluding any other elements that interfere with or contribute to the activity or action of the listed elements. Thus, the phrase “consisting essentially of” indicates that the listed elements are required or mandatory, but that other elements may be included only if they do not materially affect or contribute to the activity or action of the listed elements.

The composition can be packaged within a container and provided with instructions for using the composition for one or more of the methods disclosed herein. For example, the composition can be in the form of a pill, capsule or powder and supplied to a subject within a container having instructions for practicing one or more methods disclosed herein, including dosage information as disclosed herein. In some embodiments, the composition is provided in bulk for use in the manufacture of nutritional supplement products. In such embodiments, the composition can be placed in containers suitable for the storage and distribution of the composition in bulk, such as drums, for example. Such containers can be airtight or otherwise adapted to protect the composition from degradation of one or more active ingredients of the composition due to atmospheric moisture and oxidation.

### EXAMPLES

A standardized polysaccharide extract was extracted from ginger (Zingiber officinale). The immunomodulatory activity of the extract was evaluated with in vitro cell models. Cell proliferation, cytokines, phagocytosis, and other experiments were used to prove the extract was effective on immune activation of RAW 264.7 macrophages. It was also shown that the extract exerts immunostimulatory effects via TLR-4-MAPK/NF-kB signaling pathway.

### Cell Culture and Reagents

The murine macrophage-like cell line RAW264.7 was purchased from National Centre for Cell Science, Pune, India and cultured in Dulbecco's modified Eagle's medium (DMEM) supplemented with 10% fetal bovine serum (FBS), 100 U/mL penicillin, and 100 μg/mL streptomycin at 37° C. in a 5% CO2 humidified atmosphere.

### Cell Viability Assay

To investigate the effect of the extract on cell viability, RAW 264.7 cells were plated at a density of 15×10³ cells/well in a 96 well plate and incubated overnight, cells were treated with varying concentrations (0.25-2.5 mg/mL) of the inventive extract and incubated for 24 h. Cell viability was measured by the MTT assay.

### Morphological Change of RAW264.7 Cells

RAW264.7 cells (3.5×105 cells/well) were cultured in 6-well plates and incubated for 24 h. Cells were then treated with two different concentrations of the inventive extract (50 and 100 μg/ml) or LPS (1 μg/ml) for 24 h. Morphological changes were observed using a phase-contrast microscope (magnification, 20×; Olympus Corporation).

### Measurement of Phagocytic Capacity of Macrophages in Vitro

RAW264.7 cells were incubated with the inventive extract (50 and 100 μg/mL) for 24 h, and then 100 μL of aseptic neutral red solution (0.075%) was added into each well and incubated for another 1 h. After washing with PBS, the cells were lysed by adding 150 μL cell lysis buffer (anhydrous ethanol/acetic acid, 1:1). The absorbance was measured at 550 nm using Tecan Infinite 200 Pro M Plex multimode reader. The absorbance represented the phagocytic ability of the macrophages.

### Nitric Oxide (NO) Measurement

The concentration of nitrite, an indicator of nitric oxide (NO) synthesis was measured using Griess reagent. RAW 264.7 cells were grown in 6-well plates (3.5×105 cells/mL). After 24 h, cells were treated with LPS (1 μg/mL) or the inventive extract (50 and 100 μg/mL) for 24 h. The supernatant was harvested and mixed with Griess reagent and incubated for 10 min at room temperature. Absorbance of the mixture was measured at 540 nm using Tecan Infinite 200 Pro M Plex multimode reader.

### Determination of Cytokines Production

RAW 264.7 macrophages were plated in a six-well plate (3×10⁵ cells/well). After overnight incubation, cells were treated with two different concentrations of the inventive extract (50-100 μg/ml) for 24 h. The cell supernatants were collected and assayed to determine the levels of TNF-α, IL-1βB, and IL-6) using commercial enzyme-linked immunosorbent assay (ELISA) kit (R&D systems) in accordance with the manufacturer's instruction.

### Western Blot

After being treated with the inventive extract for 24 h, the cells were lysed by RIPA buffer for 10 min and centrifuged at 12000 rpm for 10 min at 4° C. The protein concentration was measured using Bradford reagent (Bio-Rad). The samples were separated through a 12% SDS-PAGE gel and then transferred to PVDF membranes. After being blocked with 5% skimmed milk or BSA solution for 2 h, the membranes were probed with diluted primary antibodies against iNOS, COX2, TLR4, MyD88 a, p-NF-KB, p-ERK, and p-JNK in 5% BSA/TBST overnight at 4° C. After being washed with TBST and TBS 3 times, the membranes were incubated with horseradish peroxidase (HRP)-conjugated secondary antibodies for 1 h at room temperature. The blots were smeared with chemiluminescence reagent (Millipore) and analyzed by Image Quant GE Health Care.

### Statistical Analysis

Data were shown as mean±standard deviation of at least three independent experiments. One-way ANOVA and Student's t-test were used to evaluate statistical significance with the SPSS statistics 23.0 software. Values of P<0.05 were considered as statistically significant.

### Results

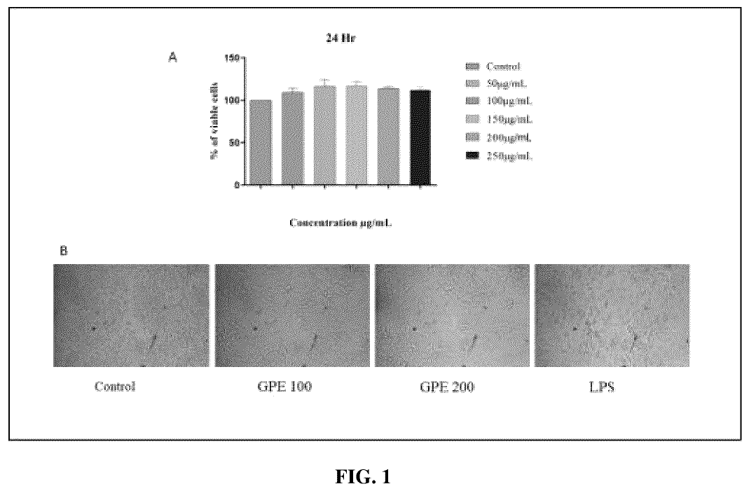

Initially, the effect of the inventive extract on mouse macrophage cell viability for 24 h of incubation was evaluated using MTT assay (FIG. 1). Following treatment with increasing concentration of the inventive extract (50-250 μg/ml), cell proliferation was 109.52, 116.66, 117.33, 114.00, and 112.33% compared with that of untreated control group (100%), which indicated that the inventive extract (50-250 μg/ml) exhibited a nontoxic effect on RAW 264.7 cells.

### Extract Enhances NO Production in RAW 264.7 Cells

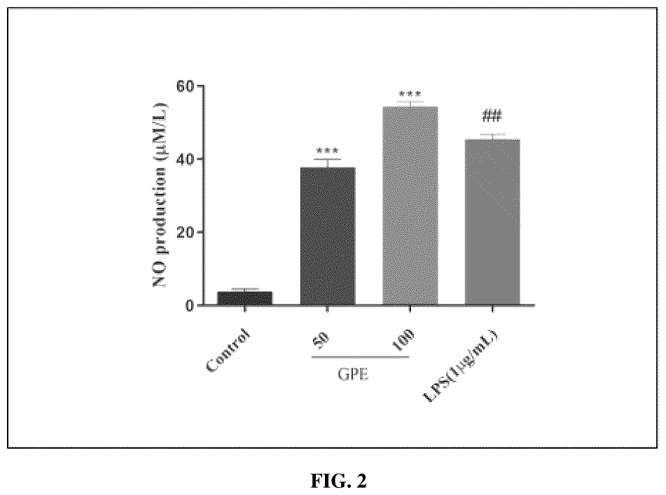

To assess the ability of the inventive extract to induce an immune response in mammals, the inventive extract was tested for its potential to stimulate NO production in RAW264.7 cells. The overall effect of the inventive extract on NO secretion was very impressive in RAW264.7 cells. As shown in FIG. 2, the inventive extract at the concentration of 50 and 100 μg/mL produced 37.-54.33 μM NO, which was significantly higher than the NO produced by control or LPS-treated RAW264.7 cells (45.41 μM), indicating that the inventive extract was able to stimulate RAW264.7 cells to the maximum.

### Extract Improves the Phagocytic Activity of RAW 264.7 Cells

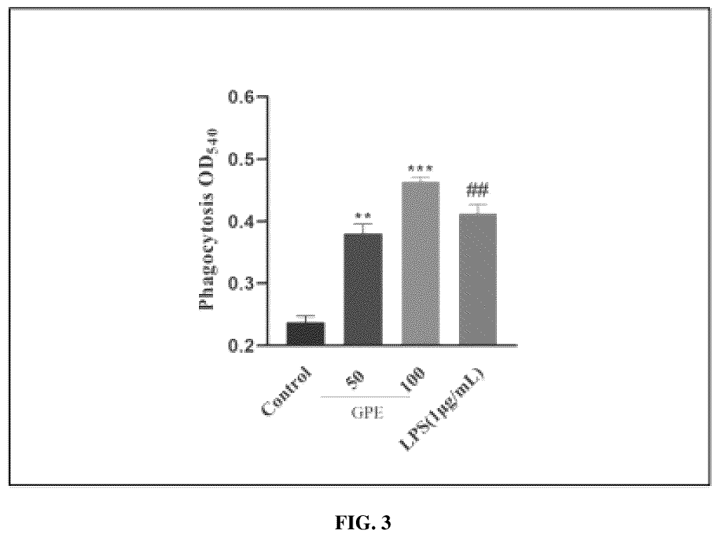

Macrophage cells have the capability of phagocytosis, which is the consumption of foreign particles. After phagocytosis, macrophages become effector cells of the humoral immune system and act as antigen-presenting cells. This helps with both adaptive and innate immunity. In our study, the effect of the inventive extract on phagocytic activity of the macrophages was determined by neutral red assay. As shown in FIG. 3, phagocytic activity was noticeably enhanced in the inventive extract treated cells (50-100 μg/ml). After 24 h treatment, the percentage of increase in the phagocytic activity with respect to untreated control (100%) was 60.33 (50 μg/ml), 95.5% (100 μg/ml) and 73.83% (LPS 1 μg/ml). Therefore, the results indicate that the inventive extract could trigger phagocytic activity of macrophage cells at lesser concentrations.

Extract increases the expression of pro-inflammatory cytokines in RAW 264.7 cells.

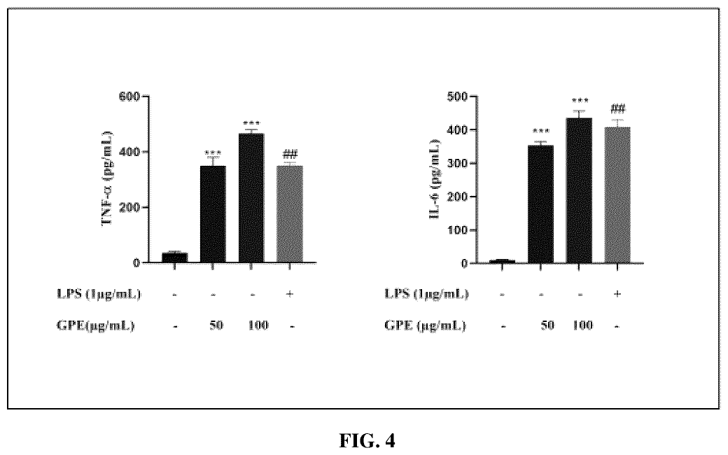

It is well-known that activated macrophages can protect against microbial infections by producing various proinflammatory cytokines (TNF-α, IL-6, etc.) which also help to eliminate invading pathogens and abnormal cells. To determine if the inventive extract could stimulate TNF-α and IL-6 production, macrophage cells were treated with two different doses (50 and 100 μg/mL) for 24 h and culture supernatants were subjected to ELISA for TNF-α and IL-6 production analysis. As shown in FIG. 4, the concentration of TNF-α and IL-6 after addition of the inventive extract increased in a dose-dependent manner. The cytokine levels increased significantly compared to untreated cells. Our results indicate that the inventive extract exerts immunostimulatory activity.

Effect of Extract on COX-2 and iNOS Protein Expression in RAW 264.7 Cells

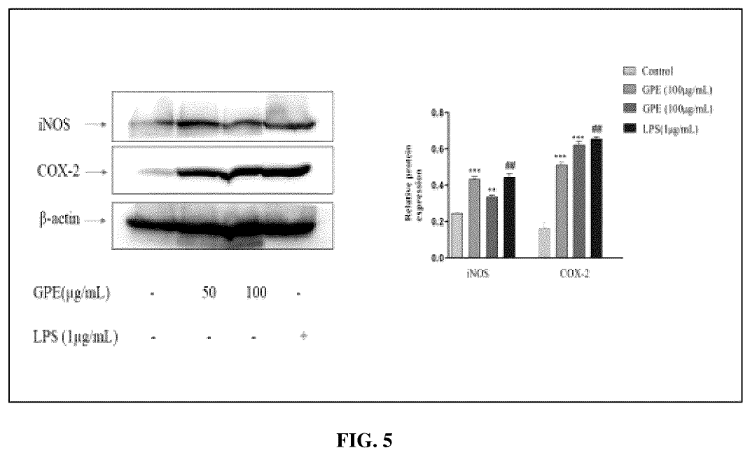

Cyclooxygenase-2 (COX-2) converts arachidonic acid to prostaglandin E2 (PGE2) and modulates immune function by producing cytokines. Inducible nitric oxide synthase (iNOS) contributes to pathogen death through NO produced by oxidizing 1-arginine, while NO exhibits immunomodulatory roles, such as T cell activity regulation. To determine the effect of the inventive extract on the activation of COX-2 and iNOS, the total protein of inventive extract treated RAW 264.7 cell was analyzed by immunoblotting. As shown in FIG. 5, COX-2 and iNOS protein expression was significantly increased by the inventive extract treatment in a dose-dependent manner.

### Extract Increases the Protein Expression in TLR4 Signaling Pathways in Macrophages

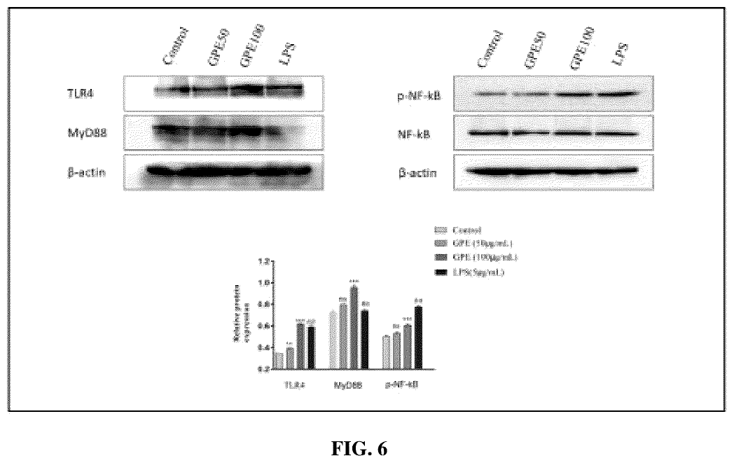

It has been documented that the TLR family, being key pattern recognition receptors, are responsive to a variety of ligands. These ligands include viral proteins, bacterial proteins, and nucleic acid fragments. TLR4 is a protein that helps the body fight infection by binding to MyD88. This activates NF-KB, which helps the body produce cytokines. Cytokines help the body fight infection. TLR4-based immunotherapy is a new way to treat cancer. The results showed that in macrophages, the inventive extract significantly induced the protein levels (FIG. 6) in the TLR4 signaling pathways (P<0.05) showing that TLR4-MyD88 may be one of the signaling pathways by which the inventive extract plays an immunomodulatory role.

### Extract Activates Mitogen-Activated Protein Kinase (MAPK) Pathway

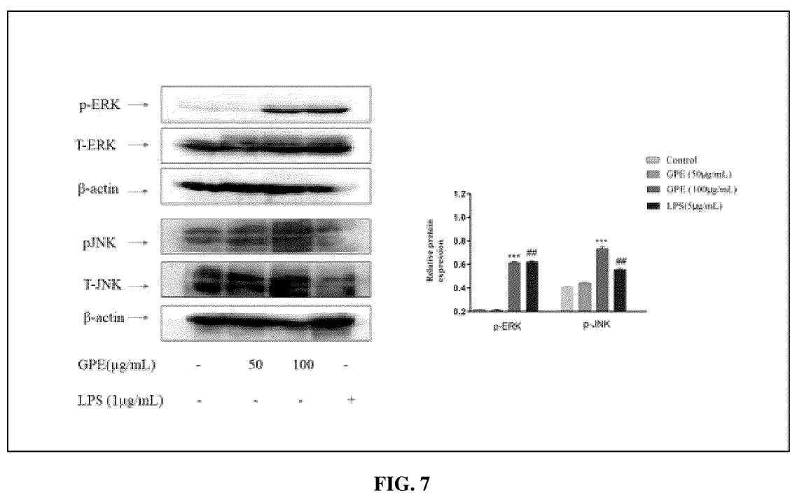

The mitogen-activated protein (MAP) kinases are intermediate proteins that mediate inflammatory signals by phosphorylation and serve to modulate macrophage activation and differentiation. Activation of MAPK including ERK, JNK, and p38 leads to upregulation of proinflammatory mediators and activation of cytokine production. It was explored whether extract stimulated macrophage activation, characterized by morphology alteration and increased production of cytokines and nitrites, is mediated via activation of MAPK signaling pathways. RAW 264.7 cells were incubated with two concentrations of the inventive extract (50 and 100 μg/mL) and the degree of phosphorylation of MAPK was examined by western blotting. When the cells were treated with the inventive extract, the levels of p-ERK and p-JNK were elevated significantly compared to control cells. However, changes in the expression of p-p38 could not be detected (FIG. 7). Notably, the levels of phosphorylated MAPK were almost similar to LPS-treated cells. These results suggest that the inventive extract activates MAPK signaling, which is associated with macrophage activation, in part.

1. A method of promoting immune system health, comprising administering to a subject an effective amount of a composition comprising ginger extract, wherein the ginger extract comprises one or more polysaccharides.

2. The method of claim 1, wherein the ginger extract is an extract of Zingiber officinale.

3. The method of claim 1, wherein the ginger extract comprises an extract of ginger root.

4. The method of claim 1, wherein the ginger extract is administered systemically or locally.

5. The method of claim 1, wherein the ginger extract is administered orally.

6. The method of claim 5, wherein the composition is in the form of a powder, liquid, pill, tablet, pellet, capsule, thin film, solution, spray, syrup, linctus, lozenge, pastille, chewing gum, paste, vapor, suspension, bead, gummy, gel, or sol.

7. The method of claim 5, wherein the composition is in the form of a tea.

8. The method of claim 1, wherein the composition is administered topically.

9. The method of claim 8, wherein the composition is in the form of a powder, thin film, solution, spray, syrup, paste, vapor, suspension, emulsion, ointment, cream, lotion, drop, topical patch, gel, or sol.

10. The method of claim 1, wherein the subject is infected with a pathogen.

11. The method of claim 10, wherein the subject is experiencing one or more symptoms of being infected with a pathogen.

12. The method of claim 11, wherein the symptoms include coughing, fever, chills, headache, nasal congestion, runny nose, sneezing, muscle aches, fatigue, irritated respiratory tract, irritated throat, diarrhea, or combinations thereof.

13. The method of claim 1, wherein the subject is a human.

14. The method of claim 1, wherein the ginger extract is obtained by aqueous solvent extraction, organic solvent extraction, supercritical fluid solvent extraction, or combinations thereof.
